(19) United States
(12) Patent Application Publication
(10) Pub. No.: US 2024/0050431 A1
(43) Pub. Date: Feb. 15, 2024

(54) NEUROENDOCRINE DIFFERENTIATION IN IMIPRIDONE CANCER TREATMENT

(71) Applicant: BROWN UNIVERSITY, Providence, RI (US)

(72) Inventors: Wafik S. El-Deiry, Barrington, RI (US); Elizabeth Ding, Providence, RI (US)

(21) Appl. No.: 18/230,810

(22) Filed: Aug. 7, 2023

Related U.S. Application Data
(60) Provisional application No. 63/395,695, filed on Aug. 5, 2022.

(51) Int. Cl. A61K 31/519; A61P 35/00; G01N 33/574

(52) U.S. Cl. CPC A61K 31/519; A61P 35/00; G01N 33/57484

(57) ABSTRACT

A method of treating a neuroendocrine tumor in a subject is described. The method includes determining the level of expression of neuroendocrine differentiation markers and/or TRAIL signaling pathway markers by the tumor, and either treating the subject with a therapeutically effective amount of an imipridone compound if the level of expression of one or more markers is high or treating the subject with a higher dose of the imipridone compound or a different neuroendocrine tumor treatment method.

### CROSS-REFERENCE TO RELATED APPLICATIONS

This application claims priority to U.S. Provisional Application Ser. No. 63/395,695, filed on Aug. 5, 2022, which is hereby incorporated by reference in its entirety.

### BACKGROUND

Neuroendocrine cancer is a relatively rare and heterogeneous tumor type. Neuroendocrine tumors (NETs) harbor neuroendocrine differentiation (ND) with specific markers including protein gene product 9.5 (PGP9.5) and Chromogranin A (CgA). In prostate cancers (PC), ND is induced by BRN2/SOX2 transcription factors. NET-like cells with low or absent androgen receptor (AR) signaling cause hormone therapy resistance and poor prognosis in PC. Small cell lung carcinoma (SCLC), a high-grade NET, presents with metastasis early and has poor survival. ONC201/TIC10 is a small molecule inducer of TRAIL signaling in clinical trials. ONC201 antagonizes dopamine D2 or D3 receptors (DRD2/DRD3) and is an agonist of mitochondrial caseinolytic protease P (ClpP) resulting in activation of DR5/TRAIL-dependent apoptosis involving the integrated stress response (ISR). ONC201 is active in various malignancies including H3K27M-mutated glioma and NETs expressing high levels of DRD2.

There are currently no established biomarkers for patient selection for treatment of neuroendocrine tumors by ONC201/TIC10, other than as a group of tumors that include pheochromocytomas and paragangliomas where the tumors express high levels of certain dopamine receptors such as DRD2 or DRD3. However, these markers are neither predictive of response to the drug nor used for patient selection for patients with neuroendocrine tumors. Accordingly, there remains a need for methods of guiding treatment of neuroendocrine tumors using imipridone compounds such as ONC201.

### SUMMARY

The inventors were able to determine that the investigated cell lines show neuroendocrine differentiation markers of interest, and that ONC201 is sensitive in tumor cell lines. Based on this, a method of treating a neuroendocrine tumor in a subject was developed. The method includes determining the level of expression of neuroendocrine differentiation markers and/or TRAIL signaling pathway markers by the tumor, and either treating the subject with a therapeutically effective amount of imipridone compound if the level of expression of one or more markers is high, or treating the subject with a higher dose of imipridone compound or a different neuroendocrine tumor treatment method. In some embodiments, the level of expression of neuroendocrine differentiation markers and/or TRAIL signaling pathway markers by the tumor is determined ex vivo, while in other embodiments the level of expression of neuroendocrine of the markers by the tumor is determined in vivo.

In some embodiments, the imipridone compound is selected from the group consisting of ONC201, ONC206, ONC212, ONC213, and ONC219. In further embodiments, the imipridone compound is ONC201.

In some embodiments, the neuroendocrine tumor is a prostate cancer tumor. In other embodiments, the neuroendocrine tumor is a small cell lung carcinoma tumor. In further embodiments, the neuroendocrine differentiation marker is selected from PGP9.5, CgA, SOX2, and BRN2. In yet further embodiments, the TRAIL signaling pathway marker is selected from the group consisting of ATF4, DR5, ClpP, ClpX, and DRD2/DRD3.

In some embodiments, the subject is treated with an imipridone compound if the level of expression of a plurality of markers is high. In further embodiments, the subject is treated with an imipridone compound if the level of expression of one or more neuroendocrine differentiation markers is high. In yet further embodiments, the subject is treated with an imipridone compound if the level of expression of one or more TRAIL signaling pathway markers is high.

### BRIEF DESCRIPTION OF THE FIGURES

FIG. 1 provides graphs and images showing that ONC201 shows sensitivity and cytotoxicity to tumor cell lines (n=6) from prostate and small cell lung cancer tissues. Viability was assayed using CellTiter-Glo (CTG) assay and treated with ONC201 at varying drug concentrations for 72 hours.

FIGS. 2A-2C provide images showing NED marker expression in PC and SCLC. (A) Basal expression of various neuroendocrine markers and TRAIL pathway proteins were examined; (B) Protein expression of TRAIL pathway proteins were measured in DU145 (left) and 22RV1 (right) treated with ONC201 at IC50 at 12 hours, 24 hours, and 48 hour time points showed upregulation of ATF4 ISR pathway and decreased CLpX expression; (C) SiRNA knockdown of master NED transcription factor BRN2 in PC3 cell line indicated successful decrease in protein expression of BRN2 in 5, 10, and 20 pmol siRNA.

FIG. 3 provides schematics and images showing: Left: prostate and SCLC cell line classification. Investigated cell lines show potential of neuroendocrine marker expression and regulation. Right: ONC201 induces TRAIL signaling and results in cell death.

FIG. 4 provides graphs showing that protein expression quantification show the expected ONC201 cancer treatment response; Left: ATF4 expression was measured to increase in ONC201 treatment samples compared to control samples; Right: CLpX expression was measured to decrease in some ONC201 treatment samples.

### DETAILED DESCRIPTION OF THE INVENTION

The present invention provides a method of treating a neuroendocrine tumor in a subject. The method includes determining the level of expression of neuroendocrine differentiation markers and/or TRAIL signaling pathway markers by the tumor, and either treating the subject with a therapeutically effective amount of an imipridone compound if the level of expression of one or more markers is high or treating the subject with a higher dose of imipridone compound or a different neuroendocrine tumor treatment method.

### Definitions

The terminology as set forth herein is for description of the embodiments only and should not be construed as limiting of the invention as a whole. Unless otherwise specified, “a,” “an,” “the,” and “at least one” are used interchangeably. Furthermore, as used in the description of the invention and the appended claims, the singular forms “a”, “an”, and “the” are inclusive of their plural forms, unless contraindicated by the context surrounding such.

“Treat”, “treating”, and “treatment”, etc., as used herein, refer to any action providing a benefit to a patient at risk for or afflicted with a disease, including improvement in the condition through lessening or suppression of at least one symptom, delay in progression of the disease, prevention or delay in the onset of the disease, etc. Treatment also includes partial or total destruction or differentiation of the undesirable proliferating cells with minimal effects on normal cells. In accordance with the present invention, desired mechanisms of treatment at the cellular level include stimulation of differentiation in cancer and pre-cancer cells.

As used herein, the term “diagnosis” can encompass determining the likelihood that a subject will develop a disease, or the existence or nature of disease in a subject. The term diagnosis, as used herein also encompasses determining the severity and probable outcome of disease or episode of disease or prospect of recovery, which is generally referred to as prognosis). “Diagnosis” can also encompass diagnosis in the context of rational therapy, in which the diagnosis guides therapy, including initial selection of therapy, modification of therapy (e.g., adjustment of dose or dosage regimen), and the like.

“Pharmaceutically acceptable” as used herein means that the compound or composition is suitable for administration to a subject to achieve the treatments described herein, without unduly deleterious side effects in light of the severity of the disease and necessity of the treatment.

The terms “therapeutically effective” and “pharmacologically effective” are intended to qualify the amount of each agent which will achieve the goal of decreasing disease severity while avoiding adverse side effects such as those typically associated with alternative therapies. The therapeutically effective amount may be administered in one or more doses. An effective amount, on the other hand, is an amount sufficient to provide a significant chemical effect, such as the inhibition of cancer growth by a detectable amount.

The term “effective amount” as used herein refers to an amount sufficient to achieve an intended result. An “effective amount” includes an amount that is 100% effective in achieving that result, but also includes amounts that are less effective but still exhibit a significant effect. For example, an effective amount of a compound for reducing toxic side effects is an amount sufficient to reduce, but not necessarily eliminate, those effects.

A “subject,” as used herein, can be any animal, and may also be referred to as the patient. Preferably the subject is a vertebrate animal, and more preferably the subject is a mammal, such as a domesticated farm animal (e.g., cow, horse, pig) or pet (e.g., dog, cat). In some embodiments, the subject is a human.

As used herein, the term “organic group” is used to mean a hydrocarbon group that is classified as an aliphatic group, cyclic group, or combination of aliphatic and cyclic groups (e.g., alkaryl and aralkyl groups). In the context of the present invention, suitable organic groups for the compounds of this invention are those that do not interfere with the anti-cancer activity of the compounds. In the context of the present invention, the term “aliphatic group” means a saturated or unsaturated linear or branched hydrocarbon group. This term is used to encompass alkyl, alkenyl, and alkynyl groups, for example.

As used herein, the terms “alkyl”, “alkenyl”, and the prefix “alk-” are inclusive of straight chain groups and branched chain groups. Unless otherwise specified, these groups contain from 1 to 20 carbon atoms, with alkenyl groups containing from 2 to 20 carbon atoms. In some embodiments, these groups have a total of at most 10 carbon atoms, at most 8 carbon atoms, at most 6 carbon atoms, or at most 4 carbon atoms. Alkyl groups including 4 or fewer carbon atoms can also be referred to as lower alkyl groups. Alkyl groups can also be referred to by the number of carbon atoms that they include (i.e., C1-C4 alkyl groups are alky groups including 1-4 carbon atoms).

The term “haloalkyl” is inclusive of groups that are substituted by one or more halogen atoms, including perfluorinated groups. This is also true of other groups that include the prefix “halo-”. Examples of suitable haloalkyl groups are chloromethyl, trifluoromethyl, and the like. Halo moieties include chlorine, bromine, fluorine, and iodine.

The term “aryl” as used herein includes carbocyclic aromatic rings or ring systems. Examples of aryl groups include phenyl, naphthyl, biphenyl, fluorenyl and indenyl. Aryl groups may be substituted or unsubstituted.

Unless otherwise indicated, the term “heteroatom” refers to the atoms O, S, or N. The term “heteroaryl” includes aromatic rings or ring systems that contain at least one ring heteroatom (e.g., O, S, N). In some embodiments, the term “heteroaryl” includes a ring or ring system that contains 3 to 12 carbon atoms, 1 to 3 rings, 1 to 4 heteroatoms, and O, S, and/or N as the heteroatoms. Suitable heteroaryl groups include furyl, thienyl, pyridyl, quinolinyl, isoquinolinyl, indolyl, isoindolyl, triazolyl, pyrrolyl, tetrazolyl, imidazolyl, pyrazolyl, oxazolyl, thiazolyl, benzofuranyl, benzothiophenyl, carbazolyl, benzoxazolyl, pyrimidinyl, benzimidazolyl, quinoxalinyl, benzothiazolyl, naphthyridinyl, isoxazolyl, isothiazolyl, purinyl, quinazolinyl, pyrazinyl, 1-oxidopyridyl, pyridazinyl, triazinyl, tetrazinyl, oxadiazolyl, thiadiazolyl, and so on.

### Treating Neuroendocrine Tumors

A method of treating a neuroendocrine tumor in a subject, comprising determining the level of expression of neuroendocrine differentiation markers and/or TRAIL signaling pathway markers by the tumor, and either treating the subject with a therapeutically effective amount of an imipridone compound if the level of expression of one or more markers is high, or treating the subject with a higher dose of imipridone compound or a different neuroendocrine tumor treatment method.

The invention includes treating subjects having a neuroendocrine tumor with a therapeutically effective amount of an imipridone compound. The angular structure of the imipridone scaffold has been identified. Wagner et al., Oncotarget, 5 (24), 12728 (2014) and a variety of imipridone compounds have been identified. Prabhu et al., Neoplasia, 22, 12, 725 (2020). Examples of imipridone compounds that can be used for treating colon cancer include ONC201, ONC206, ONC212, ONC213, and ONC219. Bonner et al., Neuro-oncology, 23(4), 542 (2021). Imipridone compounds can be represented by formula I:

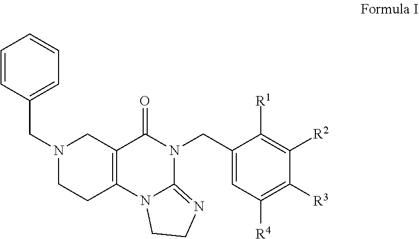

wherein R¹, R², R³, and R⁴ are independently selected from H, —CH₃, —CF₃, and halogen.

In some embodiments, the imipridone compound is selected from the group consisting of ONC201, ONC206, ONC212, ONC213, and ONC219, while in further embodiments the imipridone compound is ONC201. The structure of ONC201 is shown below:

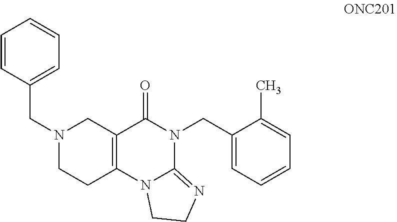 [SMILES: Cc1ccccc1CN1C(=O)C2=C(CCN(Cc3ccccc3)C2)N2CCN=C12]

As used herein, the terms “tumor” or “cancer” refer to a condition characterized by anomalous rapid proliferation of abnormal cells of a subject. The abnormal cells often are referred to as “neoplastic cells,” which are transformed cells that can form a solid tumor. The term “tumor” refers to an abnormal mass or population of cells (e.g., two or more cells) that result from excessive or abnormal cell division, whether malignant or benign, and pre-cancerous and cancerous cells. Malignant tumors are distinguished from benign growths or tumors in that, in addition to uncontrolled cellular proliferation, they can invade surrounding tissues and can metastasize.

Cancer is generally named based on its tissue of origin. There are several main types of cancer. Carcinoma is cancer that begins in the skin or in tissues that line or cover internal organs. Sarcoma is cancer that begins in bone, cartilage, fat, muscle, blood vessels, or other connective or supportive tissue. Leukemia is cancer that starts in blood-forming tissue such as the bone marrow, and causes large numbers of abnormal blood cells to be produced and enter the bloodstream. Lymphoma and multiple myeloma are cancers that begin in the cells of the immune system. In some embodiments, the cancer is selected from the group of cancer types consisting of sarcoma, carcinoma, and lymphoma.

Cancer can also be characterized based on the organ in which it is growing. Examples of cancer characterized in this fashion include bladder cancer, prostate cancer, liver cancer, breast cancer, colon cancer, and leukemia. Solid tumors are more associated with the formation of an immune suppressive tumor microenvironment. In some embodiments, the cancer being treated a solid tumor cancer selected from the group consisting of breast, colon, bladder, prostate, and lung cancer. The type of cancer present in a subject can be determined using diagnostic methods known to those skilled in the art. Common tests used to help diagnose different types of cancer include blood chemistry tests, complete blood counts, cytogenetic analysis, immunophenotyping, liquid biopsy, sputum cytology, tumor marker tests, urinalysis, and various imaging methods such as CT scan, MRI, ultrasound, and PET scan.

The present invention provides a method of treating a neuroendocrine tumor in a subject. Neuroendocrine tumors are cancers that begin in specialized cells called neuroendocrine cells. Oronsky et al., Neoplasia, 19(12): 991-1002 (2017). Neuroendocrine cells are present not only in endocrine glands throughout the body that produce hormones, but are found in all body tissues. Although there are many kinds of NETs, they are treated as a group of tissue because the cells of these neoplasms share common features, including a similar histological appearance, having special secretory granules, and often producing biogenic amines and polypeptide hormones. Neuroendocrine tumors include well-differentiated neuroendocrine tumors, well-differentiated neuroendocrine carcinomas, and poorly differentiated neuroendocrine carcinomas. Neuroendocrine cells have traits similar to those of nerve cells and hormone-producing cells. Neuroendocrine tumors are rare and can occur anywhere in the body, typically originating the foregut, the midgut, and the hindgut. Neuroendocrine tumors of the foregut include tumors in the thymus, esophagus, lung (e.g., small cell lung carcinoma), stomach, duodenum, and pancreas. Neuroendocrine tumors of the midgut include tumors in the appendix, ileum, cecum, and ascending colon, while neuroendocrine tumors of the hindgut include tumors of the rectum and distal bowel. Neuroendocrine tumors can also be pituitary gland, thyroid gland, parathyroid tumors, peripheral nervous system tumors, and genitourinary tract tumors (e.g., urinary tract, ovary, or prostate tumors). In some embodiments, the neuroendocrine tumor is a prostate cancer tumor, while in other embodiments the neuroendocrine tumor is a small cell lung carcinoma tumor.

A solid tumor is a malignancy that forms a discrete tumor mass of tissue that usually does not contain cysts or liquid areas. It may be any tumor of the tissue such as a sarcoma, a carcinoma, or a lymphoma. Different types of solid tumors are named for the type of cells that form them. For example, a solid tumor can be a bladder, brain, breast, prostate, lung, liver, ovarian, testicular, colorectum, or kidney tumor; or sarcoma or melanoma. In one embodiment, wherein the oxygenation image is obtained through photoacoustic imaging, the solid tumor is a solid tumor located in a tissue other than the lung. In some embodiments, the solid tumor is a glioma (i.e., a tumor forming in the brain or spine) or a solid tumor resulting from pancreatic cancer.

The presence of a solid tumor can be confirmed using a variety of techniques known to those skilled in the art. Examples of procedures suitable for determining whether a subject has a solid tumor include, but are not limited to, digital examination, cystoscopy, ultrasound, infrared spectral analysis, and magnetic resonance imaging. A preferred method for confirming the presence of a solid tumor is to obtain a biopsy. In a biopsy, a tissue sample is typically obtained from a suspect tissue region using a biopsy gun which inserts and removes special hollow-core needles. The tissue samples are then examined under a microscope to determine whether cancer cells are present, and to evaluate the microscopic features of any cancer found to further characterize and possible stage the cancer.

The method includes determining the level of expression of neuroendocrine differentiation markers and/or TRAIL signaling pathway markers by the tumor. Markers include genes, proteins, and antigens. Methods of determining the level of expression of a marker depend on whether the marker is being detected as a gene, protein, or antigen, using methods known to those skilled in the art.

Methods of determining gene expression level include, for example, quantitative RT-PCR, Northern blot, real-time PCR, PCR, allele-specific PCR, pyrosequencing, SNP Chip technology, flow cytometry incorporating RNA hybridization, Northern Blot, SNP Chip technology, or restriction fragment length polymorphism (RFLP). The term “polymerase chain reaction” (PCR) refers to methods for increasing the concentration of a segment of a target sequence in a mixture of genomic DNA without cloning or purification.

Methods of determining protein expression include the detection of antigens when immunological methods are used. The presence and/or amount of marker protein can be determined using polyclonal or monoclonal antibodies that are immunoreactive with the marker protein. Use of antibodies comprises contacting a sample taken from the individual with one or more of the antibodies; and assaying for the formation of a complex between the antibody and a protein or peptide in the sample. For ease of detection, the antibody can be attached to a substrate such as a column, plastic dish, matrix, or membrane, preferably nitrocellulose. The sample may be untreated, subjected to precipitation, fractionation, separation, or purification before combining with the antibody. Interactions between antibodies in the sample and the marker protein are detected by radiometric, colorimetric, or fluorometric means, size-separation, or precipitation. Preferably, detection of the antibody-protein or peptide complex is by addition of a secondary antibody that is coupled to a detectable tag, such as for example, an enzyme, fluorophore, or chromophore. Formation of the complex is indicative of the presence of the marker protein in the sample.

Antibodies immunospecific for the marker protein may be made and labeled using standard procedures and then employed in immunoassays to detect the presence of the marker in a sample. Suitable immunoassays include, by way of example, immunoprecipitation, particle immunoassay, immunonephelometry, radioimmunoassay (RIA), enzyme immunoassay (EIA) including enzyme-linked immunosorbent assay (ELISA), sandwich, direct, indirect, or competitive ELISA assays, enzyme-linked immunospot assays (ELISPOT), fluorescent immunoassay (FIA), chemiluminescent immunoassay, flow cytometry assays, immunohistochemistry, Western blot, and protein-chip assays using for example antibodies, antibody fragments, receptors, ligands, or other agents binding the target analyte. Polyclonal or monoclonal antibodies raised against the marker protein are produced according to established procedures. Generally, for the preparation of polyclonal antibodies, a protein or peptide fragment thereof is used as an initial step to immunize a host animal. A general review of immunoassays is available in Methods in Cell Biology v. 37: Antibodies in Cell Biology, Asai, ed. Academic Press, Inc. New York (1993), and Basic and Clinical Immunology 7th Ed., Stites & Terr, eds. (1991).

In some embodiments, the marker protein is detected using a method other than an immunoassay. For example, a marker protein can be detected using matrix-assisted laser desorption-ionization time-of-flight mass spectrometry (MALDI-TOF). The marker protein can also be detected by purifying the protein and determining its sequence using peptide sequencing methods. Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified and/or quantified using chromatographic and electrophoretic techniques to achieve partial or complete purification (or purification to homogeneity). Analytical methods particularly suited to the preparation of a pure peptide are immunohistochemistry, ion-exchange chromatography, exclusion chromatography; polyacrylamide gel electrophoresis; isoelectric focusing. A particularly efficient method of purifying peptides is fast protein liquid chromatography or even HPLC. Likewise, a variety of methods of protein sequencing are known to those skilled in the art. For example, the sequence may be identified using mass spectrometry or the Edman degradation reaction.

Once the presence and/or levels of the marker has been determined, they can be displayed in a variety of ways. For example, the levels can be displayed graphically on a display as numeric values or proportional bars (i.e., a bar graph) or any other display method known to those skilled in the art. The graphic display can provide a visual representation of the amount of the signature gene being evaluated.

Neuroendocrine differentiation markers are genes, proteins, or antigens known to be associated with the differentiation of neuroendocrine cells, and in particular neuroendocrine prostate cancer cells. Examples of neuroendocrine differentiation markers include Chromogranin A (CgA), Synaptophysin (SYP), protein gene product 9.5 (PGP9.5), Sex-determining region Y-related HMG box 2 (SOX2), Cluster of differentiation 56 (CD56), Neuron-specific enolase (NSE), Brain-Specific Homeobox Protein 2 (BRN2), ISL LIM Homeobox 1 (ISL1), INSM Transcription Repressor 1 (INSM1) and Secretagogin (SECG). Juhlin, C., Biology (Basel), 10(9), 874 (2021) and Sun et al., Am J Transl Res., 1(2):148-62 (2009). Accordingly, in some embodiments the neuroendocrine differentiation marker is selected from PGP9.5, CgA, SOX2, and BRN2.

In some embodiments, the level of expression of a TNF-related apoptosis-inducing ligand (TRAIL, also known as Apo2L) signaling pathway marker is determined. TRAIL interacts with death receptors (including TRAIL receptors) on cancer cells and activates apoptotic pathways in cancer cells, but not in healthy cells. Ashkenazi, A., Nat Rev Cancer 2(6): 420-430 (2002). TRAIL shows homology to other members of the tumor necrosis factor superfamily. It is composed of 281 amino acids and has characteristics of a type II transmembrane protein. TRAIL signaling pathway markers are genes and proteins involved in TRAIL signaling. Razeghian et al., Front Immunol., 12:699746 (2021). Examples of TRAIL signaling pathway markers include Activating transcription factor 4 (ATF4), Death Receptor 5 (DR5), Caseinolytic protease P (ClpP), Caseinolytic protease X (ClpX), and dopamine D2 or D3 receptors (DRD2/DRD3). Accordingly, in some embodiments the TRAIL signaling pathway marker is selected from the group consisting of ATF4, DR5, ClpP, ClpX, and DRD2/DRD3.

The present invention includes the use of diagnosis of a subject to guide treatment. As noted herein, “Diagnosis” can also encompass diagnosis in the context of rational therapy, in which the diagnosis guides therapy, including initial selection of therapy, modification of therapy (e.g., adjustment of dose or dosage regimen). The invention can be used to guide treatment of neuroendocrine cancer based on the presence of markers in the tumor, thereby determining if an imipridone compound will be therapeutically effective, or if a different therapy, or a higher dose of an imipridone compound, will be required.

The method includes treating the subject with an imipridone compound if the level of expression of one or more markers (i.e., neuroendocrine differentiation markers and/or TRAIL signaling pathway markers) is high. A high level of expression is one in which the expression level is higher than that of a control value. Where more than one marker is being evaluated, the marker levels are compared with the control levels for the appropriate corresponding markers. Control levels represent the level of the marker in a typical or average tumor or subject. Control levels may be available in the literature, or they may be determined by evaluating marker levels from a pool of tumors or subjects. In some embodiments, a higher level of expression is 10%, 20%, 30%, 40%, 50%, 60%, 70%, 80%, 90%, 100%, 150%, or more than 200% higher than the control level.

The comparison can be conducted by any suitable method known to those skilled in the art. For example, the comparison can be carried out mathematically or qualitatively by an individual operating the analytic device or by another individual who has access to the data provided by the analytic device. Alternately, the steps of determining and comparing the levels of markers can be carried out electronically (e.g., by an electronic data processor).

The level of expression of the neuroendocrine differentiation markers and/or TRAIL signaling pathway markers by the tumor can be determined either in vivo or ex vivo. In some embodiments, level of expression of neuroendocrine differentiation markers and/or TRAIL signaling pathway markers by the tumor is determined ex vivo. When determined ex vivo, a sample of the tumor being treated is removed from the subject, and then the level of markers in the tumor is evaluated using the methods described herein. Alternately, or in addition, a biological sample is obtained that includes a significant number of neuroendocrine differentiation markers and/or TRAIL signaling pathway markers expressed by the tumor, which is then evaluated using the methods described herein.

A “biological sample,” as used herein, is meant to include any biological sample from a subject that is suitable for analysis for detection of the of the marker genes or their associated RNA or proteins. Suitable biological samples include but are not limited to bodily fluids such as blood-related samples (e.g., whole blood, serum, plasma, and other blood-derived samples), urine, sputem, cerebral spinal fluid, bronchoalveolar lavage, and the like. Another example of a biological sample is a tissue sample. In some embodiments, the biological sample is a cancer cell or tissue including cancer cells.

The methods involve providing or obtaining a biological sample from the subject, which can be obtained by any known means including needle stick, needle biopsy, swab, and the like. In an exemplary method, the biological sample is a blood sample, which may be obtained for example by venipuncture.

In some embodiments, the level of expression of neuroendocrine differentiation markers and/or TRAIL signaling pathway markers by the tumor is determined in vivo. When in vivo detection of markers is performed, an in vivo diagnostic compound is introduced into the body to elicit a response which indicates the level of level of expression of a neuroendocrine differentiation marker and a TRAIL signaling pathway markers. For example, nanoparticles bearing antibodies or polynucleotides can be used for in vivo diagnostic testing.

In some embodiments, the subject is treated with an imipridone compound if the level of expression of a plurality of markers is high. The number of markers having a high level of expression can be 2, 3, 4, or 5 or more. In some embodiments, the level of expression of a neuroendocrine differentiation marker and a TRAIL signaling pathway marker are both higher. In some embodiments, the subject is treated with an imipridone compound if the level of expression of one or more neuroendocrine differentiation markers is high. In further embodiments, the subject is treated with an imipridone compound if the level of expression of one or more TRAIL signaling pathway markers is high.

If a higher level of expression of a neuroendocrine differentiation marker and/or a TRAIL signaling pathway marker is not detected, the subject is instead treated with a higher dose of imipridone compound or a different method of treating a neuroendocrine tumor. A higher dose of an imipridone compound is a dose that is higher than the normal therapeutically effective amount for treatment of a neuroendocrine tumor. The higher dose can be 10%, 20%, 30%, 40%, 50%, 60%, 70%, 80%, 90%, 100%, 150%, or more than 200% higher than the normal therapeutically effective dose.

A wide variety of methods other than treatment with imipiridone compounds (i.e., different methods) are known for cancer treatment. These methods can be used as an alternative to imipridone treatment, or in some embodiments can be used to supplement imipridone treatment. Methods of cancer treatment include radiation therapy, chemotherapy, radiofrequency ablation, cryoablation, thermal ablation, electroporation, alcohol ablation, high intensity focused ultrasound, photodynamic therapy, chimeric antigen receptor (CAR) T-Cell therapy, administration of monoclonal antibodies, and administration of immunotoxins. Cancer treatment can be used for both prophylactic and therapeutic treatment.

The effectiveness of cancer treatment may be measured by evaluating a reduction in tumor load or decrease in tumor growth in a subject in response to the administration of the modified immune suppressor cells. The reduction in tumor load may be represent a direct decrease in mass, or it may be measured in terms of tumor growth delay, which is calculated by subtracting the average time for control tumors to grow over to a certain volume from the time required for treated tumors to grow to the same volume.

### Administration and Formulation

The present invention also provides pharmaceutical compositions that include an imipridone or other anticancer compound as an active ingredient, and a pharmaceutically acceptable carrier or carriers, in combination with the active ingredient.

The compounds can be administered as pharmaceutically acceptable salts. Pharmaceutically acceptable salt refers to the relatively non-toxic, inorganic and organic acid addition salts of the compounds. These salts can be prepared in situ during the final isolation and purification of the compounds, or by separately reacting purified compounds with a suitable counterion, depending on the nature of the compound, and isolating the salt thus formed. Representative counterions include the chloride, bromide, nitrate, ammonium, sulfate, tosylate, phosphate, tartrate, ethylenediamine, and maleate salts, and the like. See for example Haynes et al., J. Pharm. Sci., 94, p. 2111-2120 (2005).

The pharmaceutical compositions include imipridone compounds together with one or more of a variety of physiological acceptable carriers for delivery to a patient, including a variety of diluents or excipients known to those of ordinary skill in the art. For example, for parenteral administration, isotonic saline is preferred. For topical administration, a cream, including a carrier such as dimethylsulfoxide (DMSO), or other agents typically found in topical creams that do not block or inhibit activity of the peptide, can be used. Other suitable carriers include, but are not limited to, alcohol, phosphate buffered saline, and other balanced salt solutions.

The formulations may be conveniently presented in unit dosage form and may be prepared by any of the methods well known in the art of pharmacy. Preferably, such methods include the step of bringing the active agent into association with a carrier that constitutes one or more accessory ingredients. In general, the formulations are prepared by uniformly and intimately bringing the active agent into association with a liquid carrier, a finely divided solid carrier, or both, and then, if necessary, shaping the product into the desired formulations. The methods of the invention include administering to a subject, preferably a mammal, and more preferably a human, the composition of the invention in an amount effective to produce the desired effect. The imipridone compounds can be administered as a single dose or in multiple doses. Useful dosages of the active agents can be determined by comparing their in vitro activity and the in vivo activity in animal models. Methods for extrapolation of effective dosages in mice, and other animals, to humans are known in the art; for example, see U.S. Pat. No. 4,938,949.

The compounds are preferably formulated in pharmaceutical compositions and then, in accordance with the methods of the invention, administered to a subject, such as a human patient, in a variety of forms adapted to the chosen route of administration. The formulations include, but are not limited to, those suitable for oral, rectal, vaginal, topical, nasal, ophthalmic, or parental (including subcutaneous, intramuscular, intraperitoneal, intratumoral, and intravenous) administration.

Formulations of the present invention suitable for oral administration may be presented as discrete units such as tablets, troches, capsules, lozenges, wafers, or cachets, each containing a predetermined amount of the active agent as a powder or granules, as liposomes containing the compounds, or as a solution or suspension in an aqueous liquor or non-aqueous liquid such as a syrup, an elixir, an emulsion, or a draught. Such compositions and preparations typically contain at least about 0.1 wt-% of the active agent. The amount of the compound is such that the dosage level will be effective to produce the desired result in the subject.

Nasal spray formulations include purified aqueous solutions of the active agent with preservative agents and isotonic agents. Such formulations are preferably adjusted to a pH and isotonic state compatible with the nasal mucous membranes. Formulations for rectal or vaginal administration may be presented as a suppository with a suitable carrier such as cocoa butter, or hydrogenated fats or hydrogenated fatty carboxylic acids. Ophthalmic formulations are prepared by a similar method to the nasal spray, except that the pH and isotonic factors are preferably adjusted to match that of the eye. Topical formulations include the active agent dissolved or suspended in one or more media such as mineral oil, petroleum, polyhydroxy alcohols, or other bases used for topical pharmaceutical formulations.

The tablets, troches, pills, capsules, and the like may also contain one or more of the following: a binder such as gum tragacanth, acacia, corn starch or gelatin; an excipient such as dicalcium phosphate; a disintegrating agent such as corn starch, potato starch, alginic acid, and the like; a lubricant such as magnesium stearate; a sweetening agent such as sucrose, fructose, lactose, or aspartame; and a natural or artificial flavoring agent. When the unit dosage form is a capsule, it may further contain a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac, sugar, and the like. A syrup or elixir may contain one or more of a sweetening agent, a preservative such as methyl- or propylparaben, an agent to retard crystallization of the sugar, an agent to increase the solubility of any other ingredient, such as a polyhydric alcohol, for example glycerol or sorbitol, a dye, and flavoring agent. The material used in preparing any unit dosage form is substantially nontoxic in the amounts employed. The active agent may be incorporated into sustained-release preparations and devices.

An example has been included to more clearly describe particular embodiments of the invention. However, there are a wide variety of other embodiments within the scope of the present invention, which should not be limited to the particular example provided herein.

### Example

### Neuroendocrine Differentiation (ND) in Sensitivity of Neuroendocrine Tumor (NET) Cells to ONC201/TIC10 Cancer Therapeutics

The inventors hypothesized that altered BRN2/SOX2 may impact NET apoptosis by ONC201 through the ISR and TRAIL/DR5. They analyzed the expression of PGP9.5, CgA, SOX2, BRN2, ATF4, DR5, ClpP, ClpX, and DRD2/DRD3 in PC and SCLC cell lines (N=6)−/+treatment with ONC201. Their results reveal that DU145 (IC50=3.11 μM), PC3 (IC50=3.02 μM), and LNCaP (IC50=1.33 μM) are ONC201 sensitive. H1417 SCLC expresses CgA, unlike PC3 and DU145. PGP9.5 is expressed in these lines. Additionally, PGP9.5 is expressed in PC3, DU145, H1417, and H1048 but not in LNCaP and 22RV1. BRN2 is expressed in PC3, H1417, and H1048 but not DU145, LNCaP, or 22RV1. ClpX is expressed in all 6 lines but at lower levels in SCLC. ClpP is expressed in the 6 lines. DR5 is expressed at higher levels in PC3, DU145, LNCaP, and 22RV1 PC versus H1417 and H1048 SCLC. SOX2 is expressed at high levels in H1417 cells. These results are establishing the landscape of ND in PC and SCLC lines for further experimentation and testing of the inventors' hypothesis.

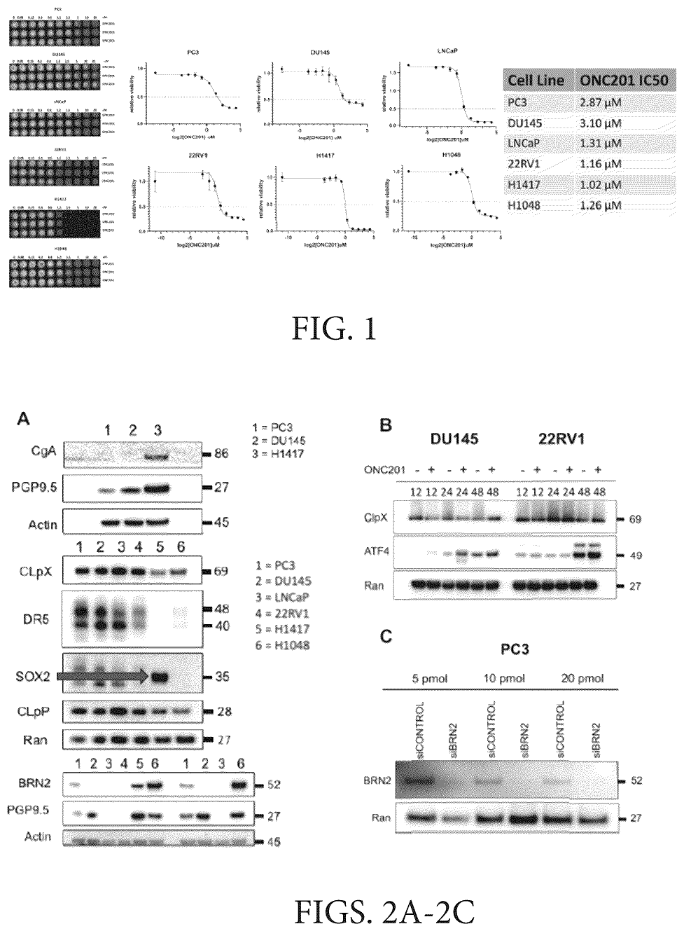

The inventors results are further supported in FIGS. 1-4. FIG. 1 provides graphs and images showing that ONC201 shows sensitivity and cytotoxicity to tumor cell lines (n=6) from prostate and small cell lung cancer tissues. Viability was assayed using CellTiter-Glo (CTG) assay and treated with ONC201 at varying drug concentrations for 72 hours.

FIGS. 2A-2C provide images showing NED marker expression in PC and SCLC. (A) Basal expression of various neuroendocrine markers and TRAIL pathway proteins were examined; (B) Protein expression of TRAIL pathway proteins were measured in DU145 (left) and 22RV1 (right) treated with ONC201 at IC50 at 12 hours, 24 hours, and 48 hour time points showed upregulation of ATF4 ISR pathway and decreased CLpX expression; (C) SiRNA knockdown of master NED transcription factor BRN2 in PC3 cell line indicated successful decrease in protein expression of BRN2 in 5, 10, and 20 pmol siRNA.

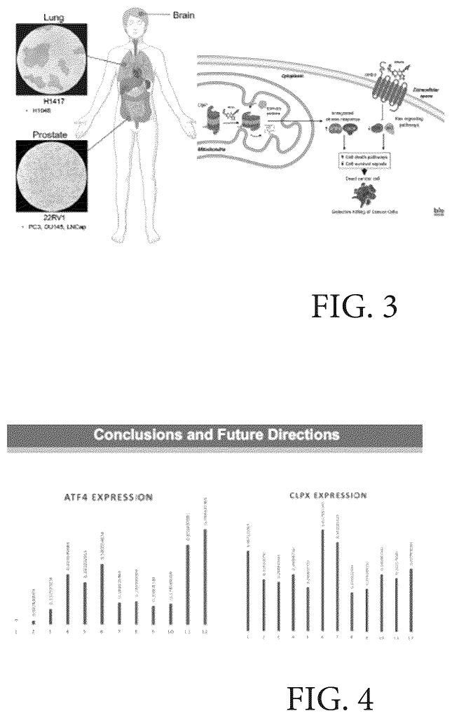

FIG. 3 provides schematics and images showing prostate and SCLC cell line classification. Investigated cell lines show potential of neuroendocrine marker expression and regulation. ONC201 induces TRAIL signaling and results in cell death.

FIG. 4 provides graphs showing that protein expression quantification show the expected ONC201 cancer treatment response; Left: ATF4 expression was measured to increase in ONC201 treatment samples compared to control samples; Right: CLpX expression was measured to decrease in some ONC201 treatment samples.

The investigated cell lines show neuroendocrine differentiation markers of interest. In addition, the inventors demonstrated that tumor cell lines were sensitive to ONC201.

The complete disclosure of all patents, patent applications, and publications, and electronically available material cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A method of treating a neuroendocrine tumor in a subject, comprising determining the level of expression of neuroendocrine differentiation markers and/or TRAIL signaling pathway markers by the tumor, and either treating the subject with a therapeutically effective amount of imipridone compound if the level of expression of one or more markers is high, or treating the subject with a higher dose of imipridone compound or a different neuroendocrine tumor treatment method.

2. The method of claim 1, wherein the imipridone compound is selected from the group consisting of ONC201, ONC206, ONC212, ONC213, and ONC219.

3. The method of claim 1, wherein the imipridone compound is ONC201.

4. The method of claim 1, wherein the neuroendocrine differentiation marker is selected from PGP9.5, CgA, SOX2, and BRN2.

5. The method of claim 1, wherein the TRAIL signaling pathway marker is selected from the group consisting of ATF4, DR5, ClpP, ClpX, and DRD2/DRD3.

6. The method of claim 1, wherein the neuroendocrine tumor is a prostate cancer tumor.

7. The method of claim 1, wherein the neuroendocrine tumor is a small cell lung carcinoma tumor.

8. The method of claim 1, wherein the subject is treated with an imipridone compound if the level of expression of a plurality of markers is high.

9. The method of claim 1, wherein the subject is treated with an imipridone compound if the level of expression of one or more neuroendocrine differentiation markers is high.

10. The method of claim 1, wherein the subject is treated with an imipridone compound if the level of expression of one or more TRAIL signaling pathway markers is high.

11. The method of claim 1, wherein the subject is human.

12. The method of claim 1, wherein the level of expression of neuroendocrine differentiation markers and/or TRAIL signaling pathway markers by the tumor is determined ex vivo.

13. The method of claim 1, wherein the level of expression of neuroendocrine differentiation markers and/or TRAIL signaling pathway markers by the tumor is determined in vivo.
